(19) United States
(12) Patent Application Publication
(10) Pub. No.: US 2023/0147961 A1
(43) Pub. Date: May 11, 2023

(54) Implantable Transition Micro-Electrodes

(71) Applicant: University of Utah Research Foundation, Salt Lake City, UT (US)

(72) Inventors: Yantao Fan, Salt Lake City, UT (US); Christopher F. Reiche, Salt Lake City, UT (US); Florian Solzbacher, Salt Lake City, UT (US)

(21) Appl. No.: 17/920,695

(22) PCT Filed: Apr. 15, 2022
(86) PCT No.: PCT/US22/25057

§ 371 Date: Oct. 21, 2022

Related U.S. Application Data
(60) Provisional application No. 63/225,826, filed on Jul. 26, 2021.
(60) Provisional application No. 63/175,400, filed on Apr. 15, 2021.

(51) Int. Cl. A61B 5/00; A61B 5/262

(52) U.S. Cl. CPC A61B 5/0004; A61B 5/262

(57) ABSTRACT

A transition microelectrode (108) can include a micro well array (104) having a plurality of microwells. The transition microelectrode (108) can further include a plurality of neuronal soma oriented within the plurality of microwells. A bioerodible probe guide (106) can be oriented over the microwell array (104). An electrode (103) can be electrically connected with the plurality of microwells. A transition microelectrode array (116) can include an electrode array having a plurality of the transition microelectrodes (108).

### RELATED APPLICATIONS

This application claims priority to U.S. Provisional Patent Application No. 63/175,400, filed Apr. 15, 2021, and U.S. Provisional Patent Application No. 63/225,826, filed Jul. 26, 2021, which are each incorporated herein by reference.

### BACKGROUND

Neural circuits determine the brain's functions and include approximately 85 billion neuronal cells. Current brain recording technology is not sufficient to accomplish the goal of a high-resolution mapping of brain activity due to the lack of a large-scale recording technology. Another challenge for current brain recording technology is obtaining longer lifetime for the implanted electrodes to prevent repeated surgeries. Over time, the harsh physiological environment (e.g., wet, ionic, reactive oxidizing species, immune response, etc) in the neural tissue breaks down and/or encapsulates the electrode implants and eventually rendering them non-functional.

Underlying neural circuits in the brain determine certain human behaviors, such as movements, memory, and cognition. Electrophysiological technologies, which include neural sensors, brain imaging, patch clamp, and optogenetics, were developed to investigate the mechanisms and functions of the brain. However, the currently available in vivo electrophysiological technologies are not sufficient to achieve large-scale brain recording with a high spatial and temporal resolution. Patch clamps allow single cell recording, but cannot be applied in a high-throughput manner in vivo. Optogenetics is associated with side effects from applying viral agents in the brain, as well tissue damage during deep brain recordings. In electrophysiology, the in vivo durability of the currently available microelectrodes is not yet compatible with the human lifespan. Due to the harsh environment in the brain, current electrode implants undergo damage to the dielectric insulation. This damage leads to exposure of conductive materials and impedance reduction over time, which limits the functional lifetime and clinical viability of neuroprosthetics. Despite the considerable resources and major efforts that were spent on searching and testing microelectrodes and encapsulation materials (e.g., parylene C, polyimide, and silicon dielectrics), the current microelectrodes still exhibit insufficient stability under a physiological environment. To date, no microelectrode encapsulation material has been identified that can withstand ten (or more) years of exposure to in vivo environments.

### SUMMARY

To overcome these obstacles, an implantable transition micro-electrode and transition micro-electrode arrays (tMEA) for large-scale brain recording and modulation can be used. This approach can achieve a density of 10⁶ electrodes per cm² or more, which is several orders of magnitude beyond established neural recording solutions. The tMEA uses living neurons as a mechanism to mediate electrical recording and its axon guiding probes can be fabricated from degradable biopolymer. The biocompatibility of the tMEA's design by integrating living neurons and soft biopolymers can greatly decrease tissue damage and may suppress inflammatory immune response in the brain. The tMEA technology can use biopolymers that degrade safely after implantation, exposing living neural stem cells that project their axons into local brain regions to form synaptic connections with the patient's own neurons. Projection can be guided by the axon guiding probes. In this way, the biological neuronal axons can grow into a long-term stable electrode array and can act as a high-performance brain-machine interface. Each of these distinctive features endow the tMEA with unique potential for neuroscientists and clinicians to explore human brain functions and treat neurological disease, enabling an advancement of neuroscience, medical practice, and a variety of other future technologies.

A transition microelectrode can comprise a microwell array having a plurality of microwells. The transition microelectrode can further include a plurality of neuronal soma oriented within the plurality of microwells. A bioerodible probe guide can be oriented over the microwell openings of the array. An electrode can be electrically connected with the plurality of microwells.

In one example, the plurality of microwells is oriented in a 2D array having microwell openings oriented toward the bioerodible probe guide. Although the number of microwells can vary considerably, in one example, the 2D array is a 10×10 regular array. Hereinafter, a regular array is defined as an aligned array wherein the rows and columns are lined up as a grid. Other suitable configurations of arrays can include, but are not limited to, honeycomb arrays, offset arrays, irregular arrays, etc.

As an example, a single neuronal soma can be oriented within each of the plurality of microwells. Accordingly, the plurality of microwells can typically be sized to accommodate a single neuronal soma. Generally, the microwells can each have a width and depth of about 3 μm to 100 μm, and in some cases 10 μm to 40 μm, and in other cases about 20 μm.

The neuronal soma can be neuronal stem cells, embryonic neural stem cells (ESCs), neural precursor cells (NPCs), induced pluripotent stem cells (iPS), and the like. As one example, the soma can be a neuronal stem cell that arises from a subject's genome. Furthermore, these soma can vary in genetic profile across the array, or remain uniform. More specifically, the neuronal soma may all have a common genetic profile, or the neuronal soma can have heterogeneous genetic profiles. In another example, the soma can have a manipulated genome to achieve selective and/or promiscuous binding.

In order to facilitate clear electrical signals from the neuronal soma to an external recording device, the plurality of microwells can include a conductive metal coating on an inner surface which is adjacent the neuronal soma. Non-limiting examples of suitable metal coating materials can include gold, platinum, iridium oxide, silicon carbide (SiC), parylene c-f coatings, carbon-based coatings (e.g. amorphous carbon, graphite, etc.), conductive polymers (e.g. PEDOT), combinations thereof, and the like. These coatings can also be optionally functionalized. As an example, the microwells can be formed and structured in conductive silicon, while a subset of microwells or each microwell can be electrically insulated from other microwells. As a general guideline, the neuronal soma can grow into the inner surface over a short period of time (e.g. a few hours). As one example, the inner surface can be roughened to increase adherence and bonding with such neuronal growth. As another example, the inner surface can be coated with a biofilm layer to provide nourishment to the soma. A biofilm layer can be used in another example to protect the electrode from oxidative stress. Non-limiting examples of such protective coatings can include parylene C and F.

In another example, the plurality of microwells can further include extracellular matrix hydrogel.

By selecting a bioerodible materials that have similar mechanical properties as targeted neural tissue, the tMEA is also mechanically more compatible thus avoiding or reducing inflammation and other immune system response after implantation. Since the property of biopolymer can be designed, degradability and degradation rate of the probe guide is controllable. The bioerodible probe guide can be formed of a bioerodible material such as a biodegradable polymer and/or a meltable material. Non-limiting examples of suitable bioerodible material can include polylactic-co-glycolic acid (PLGA), polylactic acid (PLA), polycaprolactone, polyorthoesters, aliphatic polyesters, polyhydroxybutyrate, polyglycolide (PGA), polybutylene succinate, polysaccharides, poly(ethylene glycol) (PEG), alginates, hyaluronic acids, and combinations thereof. Non-limiting examples of suitable meltable materials can include ice, and combinations thereof.

Depending on the bioerodible material, a rigidity enhancing coating can be added to increase rigidity of the probe during insertion. As one example, the rigidity enhancing coating is ice. Other non-limiting examples of rigidity enhancing coatings can include sugars, proteins (e.g. silk-elastin protein polymers SELPs), and the like.

The bioerodible probe guide can generally have a shape which allows rapid insertion into biological tissue (i.e. brain tissue, peripheral nerves, etc). In one example, the bioerodible probe guide can have a tapered profile with a probe tip. For example, the bioerodible probe guide can have a conical shape extending from the microwell array. In one example, the probe tip can have an opening permitting axons from the soma to extend outside of the opening.

Additional components can be added to one or both of the microwell environment and the probe guide. Non-limiting examples of such additives can include inhibitors, anti-inflammatory agents, growth factors, nutrients, binding proteins, anti-inflammatories, anti-scarring agents, and the like. One example of the utility these additives can provide is aid with cell differentiation of the stem cell soma. By including specific additives, the morphology and neuron type of the soma can be controlled. For example, the microwells can be coated with a biofilm layer having one or more additives. Non-limiting examples of suitable nutrients which can provide nourishment to the soma can include B-27, L-glutamate, and the like. Similarly, the biofilm layer can contain binding proteins to facilitate attachment of the soma to the microwell. Non-limiting examples of suitable binding proteins can include α-actinin, poly-1-lysine protein, and the like. Further, the biofilm layer may contain proteins to prevent oxidative stress from stimulation. In another example, the biofilm layer can include proteins which aid in cell differentiation. Non-limiting examples of such proteins can include BRN2, NFkB, SOX1, SOX6, and the like.

Furthermore, topological modifications can be introduced into the microwells which facilitate and improve adhesion of soma to inner walls of the microwells. This can assist in increasing soma lifespan and long-term mechanical stability of the tMEA. As an example, a textured surface can be formed on the inner walls, or a portion thereof. The textured surface can be a regular pattern or can be amorphous indentations to facilitate attachment of the soma to the plurality of microwells. These textured surfaces can be formed directly during manufacture of the microwells, e.g. via printing, molding, etc, or formed subsequently through surface modification such as etching, ablation, etc.

The electrode associated with the microelectrode also has the ability to transmit and receive information to and from a central computer unit. Such communication can operate in series or in parallel. Non-limiting examples of such information can include measurements of signals from the soma and variable instructions for stimulation of the soma.

In one example, the electrode comprises multiple secondary electrodes to form a composite electrode. These secondary electrodes can correspond to a subset of the soma in the array. The subsets in one example are of equal size, while in another they are of not of equal sizes. These subsets can correspond to varying genetic profiles of the soma. The subsets can be entirely distinct from one another, or have overlap and redundancy among adjacent subsets. In some cases, the subsets can equally contribute to a corresponding electrode reading, or may contribute differentially (e.g. by weighting signals differently).

A transition microelectrode array can comprise an electrode array having a plurality of the transition microelectrodes described above (see FIG. 1d).

In one example, the electrode array is a 2D array. For example, the 2D array can include a 10×10 array of the transition microelectrodes to form a composite array having 100 bioerodible probe guides and 10,000 neuronal microwells.

The plurality of transition microelectrodes can be separated by an electrically insulating material, e.g. glass. The electrodes of each transition microelectrode can be individually addressable or several can be electrically connected.

A method by which this transition microelectrode array can interface with a subject's brain can include inserting the transition microelectrode into a brain. Upon insertion in the brain, the soma project axons outside the probe guide and interface with the native neurons within the brain. This interfacing can permit stimulation and/or recording of the native cells, dependent on the instructions received by the electrodes.

In one example, the axons travel through a cavity created by the probe guide and outside the probe tip into the in vivo environment.

Projection of the axons can result in selective and/or promiscuous binding to the native cells. In one example, the previously mentioned growth factors play a role in promoting the projection of the axons. Further, an additive such as guidance molecules can determine how the axons interface with the native cells. The nature of the connection and the specificity in which native cells are interfaced with can vary dependent on the additives. The interfacing can also be determined by the genetic profile of the neurons in another example.

Although the rate of degradation can vary, once the probe guide has degraded only the axons of the neurons protrude into the neural tissue. As a result, permanent tissue scaring caused by micromovements and immune response that are typical for more rigid traditional chronic devices, particularly for deep brain implantation and recording, can be significantly reduced or eliminated. Probe guide degradation times can range generally from minutes to months, depending on the location of insertion, type of neural tissue, axon projection maturity (i.e. within the probe guide), and other factors. However, it is often desirable for the probe guide to completely degrade within about 4-6 weeks.

In one example, each soma connects to precisely one native cell, while in another the soma connect to multiple native cells. The type of synapse made in this connection can vary. Non-limiting examples include electric, chemical, mossy, filopodial, and en passant. This synapse can be excitatory in some examples, and inhibitory in others. The type of induced firing in either the native cell or the axon can be in patterns that vary as well. Non-limiting examples include standard tonic, bursting, adaptive, and delayed spike patterns.

In some examples, the number of soma stimulated varies directly with a variable input to the array.

In some examples, multiple soma can connect to the same native cell. This can permit for redundancy that allows the device to function even as some soma and axons degrade.

The electrodes in the array can measure the recordings from the soma in an aggregate, variable, and continuous manner, before the aggregated signal gets transmitted to a central unit.

In one example, the stimulating of native cells includes release of neurotransmitters for inhibition and/or excitation. Stimulating of the plurality of axons can also include a variable input to activate a variable number of soma. Such variable input can include spike patterns including one or more of standard tonic, bursting, adaptive, and delayed.

Similarly, the recording of signals from the axons can result from retrograde signaling from the axon to the soma.

Generally, each electrode can receive a plurality of recordings. Regardless, the stimulations to the native cells can be governed by a central processing unit and the signals from the native cells are transmitted as recordings to the central processing unit. These signals can be voltage signals that are variable and continuous rather than binary.

There has thus been outlined, both broadly and with specific optional elements, some features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A-1E are schematics of the Transition Micro-Electrode Array (tMEA) system. This example tMEA device is a sandwich structure, which is composed of 3 parts: a microelectrode-circuit recording system that has up to 10K recording spots, a neuron microwell (μWell) layer that has an array of up to 10K neuron microwells, and a biodegradable array of 100 axon-directing probes. In this illustration, 10K living neural stem cells are integrated into the 100×100 μwells. The 10K living neurons are individually recorded by the 10K recording spots of the recording system correspondingly. Each tMEA penetrating probe is linked to 100 μwells and can have 100 interior neuronal axons. In addition, these probes are made of biodegradable materials. Therefore, these probes can safely degrade over time in vivo, leaving only the neuron's axons projecting into the brain and connecting with the brain's local neural circuits.

FIG. 2 is a flow diagram illustrating one example method of using a transition microelectrode array in accordance with one example.

FIG. 3A is a schematic image of the tMEA chip. The tMEA chip can include peripheral bonding pads which are associated with one or more corresponding electrode wells.

FIG. 3B illustrates a set of two hybrid perishable needles each integrated with a fabricated μWell that holds a live neuron (note each probe covers an array of such single μWells that are recorded by gold electrodes). After implantation, the polymer probes form a growth pathway for the previously-stored neuronal cells in the μWell chamber.

FIG. 4A is an image of a GT2 3D lithographic 4×4 probe array.

FIG. 4B is a back side image of the array of FIG. 4A.

FIG. 4C is a side image of the array of FIG. 4A showing the bundles of wires and silicone encapsulation.

FIG. 5A. Preliminary data of the axons' projection of neurons in a 3D patterned microgel showing a bright field image of the patterned microgel.

FIG. 5B is an enlarged Calcein-AM fluorescent image of the neurons cultured in microgel from FIG. 5A.

FIG. 5C-5E are images showing the projection and development of cultured neurons' axons growing from right to left. This data demonstrated the axons can project and extend through the entire hydrogel channels.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a well” includes reference to one or more of such features and reference to “subjecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Transition Micro-Electrode Arrays

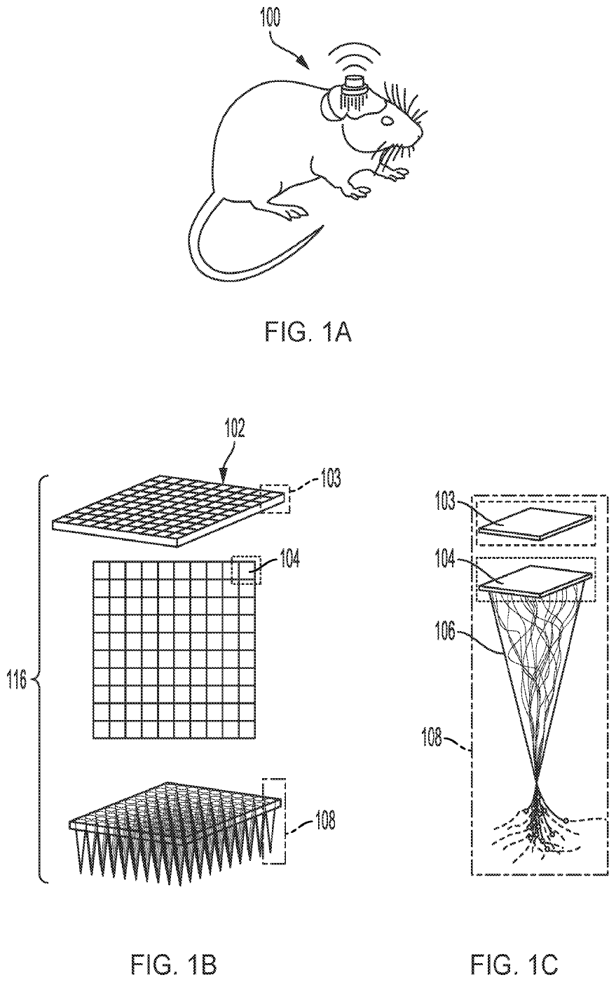

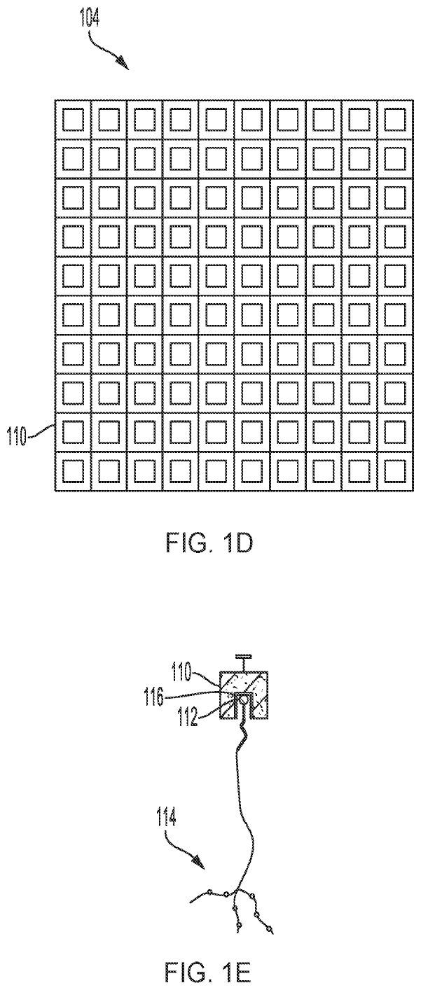

A Transition Micro-Electrode Array (tMEA) is a biocompatible brain-computer interface 100 as generally illustrated in FIG. 1A, which is composed of degradable and solid components and living neuronal cells. When implanted, the integrated neuronal cells develop their axons in the tMEA. Directed by the tMEA's biocompatible and degradable polymer probes, these axons project into the brain and form synaptic connections with the neural cells of the local neural circuits. These probes can degrade over time, eventually leaving the neuron's axons bridging the outside recording hardware with the brain's neural circuits without the probe guide. In this manner, an array of neural axons replaces and acts, together with the electronic backplane having electrically connected microwells, as microelectrodes, becoming living electrodes of the tMEA that can endure for a substantial length of time.

The tMEA can be designed to integrate living neurons for accurate large-scale recording of neural activity. With possible interface density of up to 10⁶ “electrodes” per cm² or more, tMEA technology may revolutionize microelectrode methodology and has a great potential to advance neuroscience as well as support medical treatment in the future. The tMEA uses living neurons as a mechanism of electrical recording and its axon guiding probes can be fabricated from degradable biopolymer which may be formed via 3D printing while still maintaining an ultra-high recording capability. The biocompatibility of the tMEA's design can greatly decrease tissue damage and may suppress inflammatory immune response in the brain. The tMEA technology can use biopolymers that degrade safely after implantation, exposing living neural stem cells that project their axons into local brain regions to form synaptic connections with the patient's own neurons. Projection can be guided by the axon guiding probes. In this way, the biological neuronal axons grow into a stable “electrode array” and replace a failure-prone abiotic penetrating interface with natural biotic connections which then act as a high-performance brain-machine interface. Each of these distinctive features endow the tMEA with unique potential for neuroscientists and clinicians to explore human brain functions and treat neurological disease, enabling an advancement of neuroscience, medical practice, and a variety of other future technologies.

Referring again to FIGS. 1A-1E as an example, a transition microelectrode 108 can comprise a microwell array 104 having a plurality of microwells 110. The transition microelectrode can further include a plurality of neuronal soma 112 oriented within the plurality of microwells 110. A bioerodible probe guide 106 can be oriented over the microwell openings of the array 104. An electrode 103 can be electrically connected with the plurality of microwells.

As one example, the design and fabrication of the tMEA system can use a microfabrication process that creates an electrically connected silicon microwell backplane based on the Utah array technology. The penetrating probe array can be 3D printed with suitable bioerodible materials (e.g. biopolymers) by using a two-photon polymerization process. However, other techniques can be used to form the probe array such as, but not limited to, micro-molding. The biomaterials and dimensions of these probes can be varied to optimize their biocompatibility and control the intended degradation in vivo. The biodegradable feature enables the tMEA to obtain long operational lifetime, which solves one of the major problems of conventional neural electrode arrays. The morphology and dimensions of the tMEA device can be characterized via SEM imaging. The bending and insertion can be performed by using agar-based tissue phantoms, which can be used to optimize the mechanical properties of the tMEA. Biodegradation and the estimated the lifetime of the tMEA in vivo can be evaluated via accelerated aging phosphate-buffered saline soak testing.

In one example, the plurality of microwells can be oriented in a 2D microwell array 104 having microwell openings oriented toward the bioerodible probe guide 106. Although the number of microwells can vary considerably, in one example, the 2D array is a 10×10 regular array as illustrated in FIG. 1D. Hereinafter, a regular array is defined as an aligned array wherein the rows and columns are lined up as a grid. Other suitable configurations of arrays can include, but are not limited to, honeycomb arrays, offset arrays, irregular arrays, etc. As a general guideline, the microwell array can include from 4 to 10,000 microwells, and in some cases from 36 to 1600 microwells.

Neuronal cells can be integrated with the fabricated tMEA. In an example as illustrated in FIG. 1E, a single neuronal soma 112 can be oriented within each of the plurality of microwells 110. Accordingly, the plurality of microwells can typically be sized to accommodate a single neuronal soma (approximately 3 μm to 100 μm). The cortical neural progenitor cells can be dissociated from new born rats, and culture single neuronal cells in each neuron microwell of the tMEA. Although the tMEA can typically include an array of microwells, in some cases a single microwell can be formed (as in FIG. 1E) such that the corresponding probe guide can be formed over the single microwell instead of an array of microwells.

The full device can be assembled and the entire tMEA device placed in an incubator to culture the embedded neurons. Via the direction of the polymeric probes 106, the embedded neurons 112 can project their axons 114 through the tMEA's polymer probes. Alternatively, projection of axons can occur post-implantation into a subject. In still other cases, neuronal soma can be partially cultured prior to implantation subsequent to formation of the corresponding bioerodible probe guide, and then continued projection can occur subsequent to implantation. This multi-stage growth approach can be particularly useful to axon projection beyond boundaries of the probe guide 106 (see FIG. 1C as an example).

Functional immunofluorescent staining and fluorescent imaging analysis can also provide insight into the tMEA neuronal cells' integration and development. As one example, the soma can be a neuronal stem cell that arises from a subject's genome. Furthermore, these soma can vary in genetic profile across the array, or remain uniform. By appropriate selections of neural stem cells and control of their differentiation into specific cellular phenotypes, the integrated neurons in tMEA can project into specific deep brain regions the tMEA can electrically record the brain signals from a specific region, such as prefrontal cortex (PFA), hippocampus, ventral tegmental area (VTA), among others. These long-distance but specific connections can enable tMEA spatiotemporal recording of the neural activities of specific neural circuits in a deep brain region. In this way, it is unnecessary to implant permanent monolithic electrodes into the deep brain, which can greatly decrease brain damage and side effects. Furthermore, the neuron-microwell interface determines the efficiency and function of the long-term recording. For example, 10K neurons can be integrated, although more or fewer can be included for a particular array. Regardless, this very high concentration of neurons in tMEA can ensure that the soma-circuit interface functions even if some neurons do not survive the fabrication and implantation, or otherwise degrade over time. In addition, the tMEA probe's biocompatible hydrogel environment can further increase the long-term viability of the neuron-microwell interface.

In order to facilitate clear electrical signals from the neuronal soma to an external recording device, the plurality of microwells can include a conductive metal coating on an inner surface which is adjacent the neuronal soma. Notably, in some cases the microwells can also be formed from a conductive material, e.g. highly-doped silicon. Non-limiting examples of suitable metal coating materials can include gold, platinum, iridium oxide, carbon (e.g. amorphous carbon, graphite, graphene), conductive polymers (e.g. PEDOT), and the like. Metal coating thickness can also vary based on mechanical and electrical considerations. For example, metal coating thickness can be sufficient to provide a durable mechanical interface while also providing an electrical connection. As a guideline, the metal coating thickness can range from about 5 nm to 10 μm, and often about 80 nm to 500 nm, and in some cases about 100 nm.

As a general guideline, the neuronal soma can grow into the inner surface over a short period of time (e.g. a few hours). As one example, the inner surface can be roughened to increase adherence and bonding with such neuronal growth. As another example, the inner surface can be coated with a biofilm layer to provide nourishment to the soma. A biofilm layer can be used in another example to protect the electrode from oxidative stress. Depending on the bioerodible material, a rigidity enhancing coating can be added to increase rigidity of the probe during insertion. As one example, the rigidity enhancing coating is ice. Other non-limiting examples can include parylene C and F coatings, poly(ethylene glycol) (PEG), and the like.

The bioerodible probe guide can generally have a shape which allows rapid insertion into biological tissue (i.e. brain tissue, peripheral nerves, etc). In one example, the bioerodible probe guide can have a tapered profile with a probe tip. FIG. 1C illustrates a bioerodible probe guide 106 having a conical shape with a probe tip. In one example, the probe tip can have an opening permitting axons from the soma to extend outside of the opening.

The bioerodible probe guide can be formed of a suitable material the degrades or melts over a desired degradation time. Non-limiting examples of suitable bioerodible material can include polylactic-co-glycolic acid (PLGA), polylactic acid (PLA), polycaprolactone, polyorthoesters, aliphatic polyesters, polyhydroxybutyrate, polyglycolide (PGA), polybutylene succinate, polysaccharides, poly(ethylene glycol) (PEG), conductive polymers, and combinations thereof. Non-limiting examples of suitable meltable materials can include liquid metals, ice, sugars, proteins (e.g. silk-elastin protein polymers (SELPs), and combinations thereof. The bioerodible probe guide can often be formed as a hollow shape so that axon projection can proceed within the hollow space. The hollow space can be filled with hydrogel or other suitable axon growth medium. Non-limiting examples of suitable axon growth media can include polyethylene glycol hydrogel, ECM gel, Matri-gel, gelatin methacryloyl hydrogel, and the like. There are many methods to fill these gels into the hollow space of the probes. These methods can include, but are not limited to, oxygen plasma treatment, surface modifications of the probes, and combinations thereof.

The probes themselves can be fabricated, besides 3D printing and microlithography approaches by using a blank negative of the structure fabricated from silicon using dicing and chemical wet etching techniques. Then this blank can be coated with the bioerodible polymer (spray coating, CVD) and the polymerization initialized. To open the tips of polymer shanks, laser ablation or sputtering (with an aluminum foil mask) can be used. Finally, the blank can then be removed by selective wet or dry etching methods. Alternatively, the blank can be coated with a sacrificial layer to enable removing the probe structure easier.

Additional components can be added to one or both of the microwell environment and the probe guide. Non-limiting examples of such additives can include inhibitors, anti-inflammatory agents, anti-scarring agents, and growth factors. One example of the utility these additives can provide is aid with cell differentiation of the stem cell soma. By including specific additives, the morphology and neuron type of the soma can be controlled. The immune response to implanted neurons can be avoided by implanting autologous neural stem cells or iPS cells. In addition, supporting biomaterials, such as extracellular matrix (ECM) hydrogel, can be loaded in the hollow space within the probes. Non-limiting examples of commercial ECM can include Corning Matrigel®, PuraMatrix™ Peptide Hydrogel, and the like. These gels are commercially available and are biocompatible with cell culture experiments and in vivo implantations. These supporting biomaterials provide three-dimensional (3D) structural support to neurons, and the projection of their axons. Chemicals or inhibitors for preventing inflammation and glial scarring can also be loaded along with the hydrogel. Non-limiting examples of chemical or inhibitors that may be used can include matrix metalloproteinases (MMPs), tissue inhibitors of metalloproteinases (TIMPs), TGF-β1 inhibitors, and the like. In one example, a first non-biodegradable hydrogel can be oriented within the microwells to protect the soma over time. A second biodegradable hydrogel can then be used to form the probe guide. Non-limiting examples of suitable non-biodegradable hydrogel can include chitosan hydrogels, poly(vinyl alcohol) hydrogels, and the like.

Referring back to FIG. 1C, the electrode 103 associated with the microelectrode has the ability to transmit and receive information to and from a central computer unit. Such communication can operate in series or in parallel. Non-limiting examples of such information can include measurements of signals from the soma and variable instructions for stimulation of the soma.

Most often, the transition microelectrode 108 can include a single electrode 103 for the entire microwell array 104. This means that signals from each of the microwells 110 and corresponding neurons 112 will generate a signal that is correlated with that transition microelectrode 108. However, subsets of microwells can be electrically segregated using a plurality of secondary electrodes. In one example, the electrode comprises multiple secondary electrodes to form a composite electrode. These secondary electrodes can correspond to a subset of the soma in the array. The subsets in one example are of the same size, while in another they are of varying sizes. These subsets can correspond to varying genetic profiles of the soma. The subsets can be entirely distinct from one another, or have overlap and redundancy among adjacent subsets.

Although a single transition microelectrode 108 can be useful, an array of these transition microelectrodes can also be formed. As illustrated in FIG. 1B, a transition microelectrode array 116 can comprise an array having a plurality of the transition microelectrodes 108 described above. The transition microelectrode array can generally be a planar 2D array. In one example, the 2D array can include a 10×10 array of the transition microelectrodes to form a composite array having 100 bioerodible probe guides and 10,000 neuronal microwells. However, any number of transition microelectrodes can be integrated onto the microelectrode array 116. As a general guideline, from about 16 to 2,500 microelectrodes can be included, and in some cases from 81 to 900 microelectrodes per array.

Tips of the transition microelectrodes within the array can often be formed along a uniform plane where tips each extend a uniform distance from the corresponding electrode array base. Alternatively, the tips can form a profile in which tip heights are varied across the array. As one example, tip heights can progressively increase from one edge to an opposite edge so as to form a slant array. In another example, tip heights can vary randomly or in a non-continuous pattern. Regardless, tip heights can be varied so as to allow customization of insertion depth across the array. In this manner, at least a portion of the microelectrodes can extend deeper into neural tissue than other portions of the microelectrodes. This variable tip height array can allow for sending or receiving signals from varied portions of neural tissue which would not be accessible simultaneously with an array having uniform tip heights. Regardless, the transition microelectrode array can provide deep brain stimulation and recording.

A size of the microelectrode array 116 can vary considerably. However, as a general guideline, each microelectrode array can range from about 500 μm to 10 mm across, and in some cases from 2 mm to 6 mm. Similarly, each transition microelectrode 108 can vary in size. Again, as only a general guideline, transition microelectrodes can have a width from about 30 μm to 1 mm, and often from 50 μm to 250 μm. By varying structure and dimension of the microwells, the recording spot can be fabricated to closely contact the integrated neuronal soma for well controlled extracellular recording interface.

The plurality of transition microelectrodes can be separated by an electrically insulating material, e.g. glass, ceramic, and the like. The electrodes of each transition microelectrode can be individually addressable. More specifically, in one example, each electrode 103 within an electrode array 102 can be individually addressable. In most cases, at least 90% of the electrodes 103 can be individually addressable. However, a plurality of selected transition microelectrodes can optionally be electrically connected together, e.g. through not using an insulating layer between microelectrodes or by a direct dedicated trace, wire or other electrical connection.

In still another example, the transition microelectrode array 116 can further include one or more of communication electronics, computing electronics, power source, data memory, and the like to form a biocompatible brain-computer interface 100. As one specific example, the transition microelectrode array 116 can include wired or wireless communication electronics. Non-limiting examples of such communication electronics can include Bluetooth, wireless 801 or 802 standards, micro-USB, and the like. An optional transceiver can allow for transmission and receipt of signals from a corresponding computing device. Although control of signals to individual microelectrodes can originate from a remote computing device (e.g. a handheld device, laptop, or desktop computer), an integrated computing device can be included on the transition microelectrode interface 100. Optionally, a signal processor unit can be operatively connected to receive electrical signals from the electrode. The signal processor unit can be capable of receiving information including measurements and an identifiable signature identifying the electrode as source of the information.

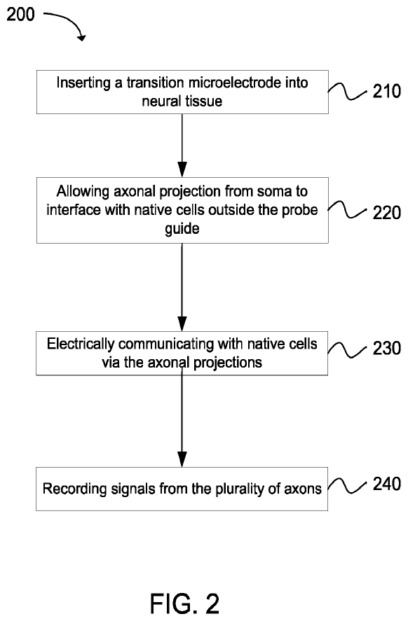

Referring now to FIG. 2, a method 200 of interfacing with neural tissue of a subject can include inserting 210 a transition microelectrode array into a region of neural tissue. Upon insertion in the brain, the soma project 220 axons outside the probe guide and interface with the native neurons within the brain. This interfacing can permit stimulation 230 and/or recording 240 of the native cells, dependent on the instructions received by the electrodes. Notably, projection of axons from the soma can include a first phase in which soma within the microwells begin axon growth which extends through the probe guide. This first phase can occur prior to insertion into neural tissue, subsequent to insertion into neural tissue, or a combination of both before and after insertion. A second phase of axon projection can involve axon growth beyond the probe guide and into surrounding neural tissue.

In one example, the axons travel through a cavity created by the probe guide, or through a medium of the probe guide, and outside the probe tip into the in vivo environment. Although conditions can vary, axon projection can generally extend from 10 nm to 100 μm from the probe guide into surrounding tissue.

By appropriate selections of neural stem cells and control of their differentiation into specific cellular phenotypes, the integrated neurons in tMEA can project into specific deep brain regions the tMEA can electrically record the brain signals from a specific region, such as prefrontal cortex (PFA), hippocampus, ventral tegmental area (VTA), among others. These long-distance but specific connections can enable tMEA spatiotemporal recording of the neural activities of specific neural circuits in a deep brain region. In this way, it is unnecessary to implant permanent monolithic electrodes into the deep brain, which will greatly decrease brain damage and side effects.

In one example, the previously mentioned growth factors play a role in promoting the projection of the axons, and in another an additive such as guidance molecules can determine how the axons interface with the native cells. The nature of the connection and the specificity in which native cells are interfaced with can vary dependent on the additives. The interfacing can also be determined by the genetic profile of the neurons in another example.

In one example, each soma connects to precisely one native cell, while in another the soma connect to multiple native cells. The type of synapse made in this connection can vary. Non-limiting examples include electric, chemical, mossy, filopodial, and en passant. This synapse can be excitatory in some examples, and inhibitory in others. The type of induced firing in either the native cell or the axon can be in patterns that vary as well. Non-limiting examples include standard tonic, bursting, adaptive, and delayed spike patterns.

In some examples, the number of soma stimulated varies directly with a variable input to the array.

In some examples, multiple soma can connect to the same native cell. This can permit for redundancy that allows the device to function even as some soma and axons degrade.

The electrodes in the array can measure the recordings from the soma in an aggregate, variable, and continuous manner, before the aggregated signal gets transmitted to a central unit.

In some cases, the neural tissue can be brain tissue, although these transition microelectrodes can also be inserted into spinal tissue and/or peripheral nervous tissue. For example, use of one or more tMEA in the brain along with complementary one or more tMEA in peripheral nervous tissue can allow for at least restoration of motor function, sensory responses, or other neural function. Neural stem cells have been shown to replenish an injured neural network. Via integrated neurons, the tMEA may provide a new and powerful tool for restoring lost sensory function, solving communication and mobility deficits, and treating neurologic diseases (e.g., stroke, paralysis, epilepsy) and neuropsychiatric disorders (including Alzheimer's and Parkinson's diseases). Such treatment can improve the quality of patients' lives and enable return to normal interaction with society. This approach integrates MEMS fabrication, stem cell biology, and 3D printing technology.

### EXAMPLE

A tMEA is integrated with 10,000 living neurons that are spatiotemporally and accurately recorded via standard microfabricated microelectrode technology. This example tMEA can be formed by combining living neurons with a 3D printed array of biodegradable, polymer-based penetrating probes to guide the axon development. While not being bound to any specific theory, these integrated living neurons appear to automatically project their axons into local brain regions, appropriately form synapses on local target neurons, and permanently integrate into the brain's neural circuits. The well-developed Utah electrode fabrication technology (UEA) provides a basis capable of connecting and recording from the neurons. Bridged by the axons of living neurons, the device can electrically receive and respond to brain signals from a specific local neural circuit. After implantation, the biocompatible polymers and stem cells in the example tMEA can greatly decrease long term tissue damage and suppress inflammatory immune response in the brain.

The design and fabrication of the example tMEA system can use a microfabrication process that creates an electrically connected silicon microwell backplane based on the Utah array technology. The penetrating probe array can be 3D printed with suitable bioerodible materials (e.g. biopolymers) by using a two-photon polymerization process. The biomaterials and dimensions of these probes can be varied to optimize their biocompatibility and control the intended degradation in vivo. The biodegradable feature enables the tMEA to obtain long operational lifetime, typically on the order of years. The morphology and dimensions of the tMEA device can be characterized via SEM imaging. The bending and insertion can be performed by using agar-based tissue phantoms, which can be used to optimize the mechanical properties of the tMEA. Biodegradation and the estimated the lifetime of the tMEA in vivo can be evaluated via accelerated aging phosphate-buffered saline soak testing.

Neuronal cells can be integrated with the fabricated tMEA for in vitro culturing and testing. The cortical neural progenitor cells can be dissociated from new born rats, and culture single neuronal cells in each neuron microwell (μWell) of the tMEA. The full device can be assembled and the entire tMEA device placed in an incubator to culture the embedded neurons. Via the direction of the polymeric probes, the embedded neurons can project their axons through the tMEA's polymer probes. The tMEA's electrochemical properties can be tested using impedance spectroscopy, cyclic voltammetry and voltage transient measurements in vitro to estimate recording function among integrated neurons within tMEA device. Functional immunofluorescent staining and fluorescent imaging analysis can also provide insight into the tMEA neuronal cells' integration and development.

Via MEMS microfabrication and metallization technology, a high-density microwell (μWell) array can be microfabricated. Each μWell can be accurately recorded by the electrode within these μWells. By integrating living neuronal cells in these μWells, the tMEA therefore allows a real-time and large-scale neural recording.

The example tMEA is a biocompatible brain-computer interface (FIG. 1A-E), which is composed of degradable components and living neuronal cells. When implanted, the integrated neuronal cells develop their axons in tMEA. Directed by the tMEA's biocompatible and degradable polymer probes, these axons project into the brain and form synaptic connections with the neural cells of the local neural circuits. These probes degrade over time, eventually leaving only the neuron's axons bridging the outside recording hardware with the brain's neural circuits (FIGS. 1A and 1E). In this manner, an array of neural axons replaces and acts as the microelectrodes, becoming living probes of the tMEA that can endure for a substantial length of time (FIG. 1E).

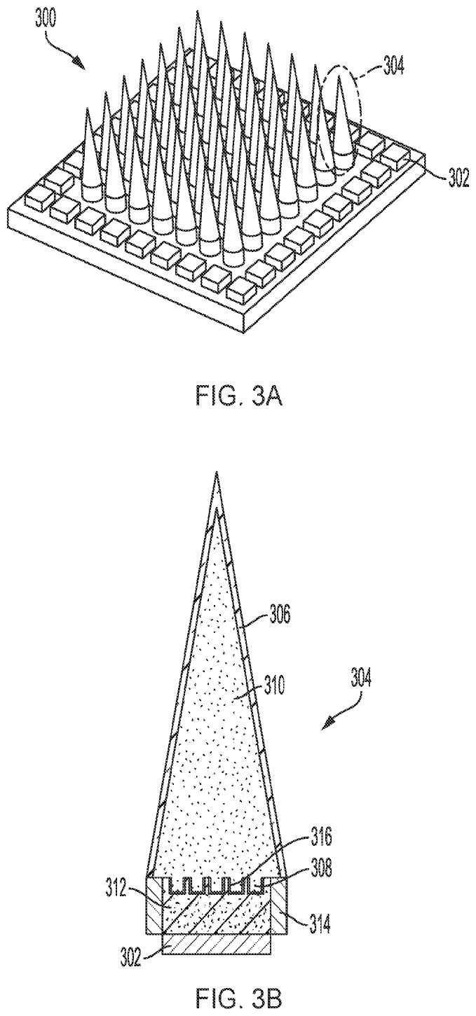

More specifically, FIGS. 3A and 3B illustrate one example of the tMEA chip 300 (i.e., the implantable part) having large-scale and micrometer-size neuroanatomical structures. A single neuron readout or bonding pad 302 can be integrated with each transition microelectrode 304 with the implantable tMEA CMOS chip 300 allows for high spatial and temporal resolution at a single-cell level.

The example tMEA device of can be equipped with 10K neuron microwells. The 100 axon-directing probes of this tMEA are designed to be linked to the neuron array. The 100 neurons that share one axon-directing probe can also share one Ti/Pt/Au recording pad 302 (FIGS. 3A and 3B). In this example, each transition microelectrode 304 includes an axon-directing probe guide 306 which is connected to an array of 100 neuron microwells 308 (i.e., a 10×10 μwell array) and is recorded on one channel through the common recording pad 302. The axon-directing probe guide 306 can include a hollow interior 310 which is filled with an axon growth medium. The axon-directing probes are designed to be biocompatible and degradable, which can first direct the neurons' axons to project into the brain and then allow the living axons to replace the prior axon-directing probes to form a permanent brain-machine interface.

In this example, a 100×100 dual-functionality microwell array can be fabricated and the inner 10K gold recording spots on a silicon substrate will store neurons and simultaneously allow electrical access to the neuron. Gold electrode coated μwells 308 are fabricated on a front side of a silicon base 312, and Ti/Pt/Au multilayer bonding pads 302 are built on a back side for electrical connection to measuring equipment. Each μWell array (i.e. collection of 308) can be isolated from adjacent μwell arrays by glass trenches 314 or other electrically insulating materials. In this way, each Ti/Pt/Au pad 302 monitors the neural signals from a group of 100 neurons that are stored in the array of 10×10 μWells 308.

As part of this example, the procedure starts from fabricating the μwell array and the recording sites. This step is a combination of wafer dicing to create trenches, melting glass into these trenches and polishing the wafer to create a wafer with an array of highly doped silicon cubes of roughly 400 μm side length. The μwells are created by a combination of lithography, deep reactive ion etching, wet chemical etching for smoothing and coating them with a Ti/Pt/Au layer. After optional surface treatment of the gold surface and a final singulation step to create individual dies, each μWell die has 10,000 μWells arranged on a 10×10 grid of recording sites on a 5×5 mm silicon/glass base with a well depth of ˜20 μm (i.e., dimension of a single neuron) and including a conductive gold coating 316 on interior surfaces of the μwells. On the other side of this silicon wafer, the Ti/Pt/Au multilayer bonding pads 302 are then deposited. After fabrication, these pads can be linked out via insulated Au wires to outside measuring equipment. The bundle of bond wires can be reinforced and isolated by medical grade silicon elastomer.

The μWell chip can be fabricated using conventional surface-micromachining techniques and can be appropriately coated with gold for the recording structures. Each bonding pad 302 can act as a recording site which can be connected to a corresponding array of μwells within a transition microelectrode 304 (see FIG. 3A), i.e., in this example, each Ti/Pt/Au pad records a group of 100 neuronal cells that are cultured in the 10×10 μWell array for that transition microelectrode (10K neurons per tMEA device). Alternatively, the number of μwells per recording site could be further reduced. For example, one μWell can be linked to one Ti/Pt/Au pad (100 neurons per tMEA chip), therefore, a single neuron recording can be formed. However, the device may also be fabricated to record each of 10K or even 1M neurons individually and simultaneously. An optional CMOS-base recording circuit can be included.

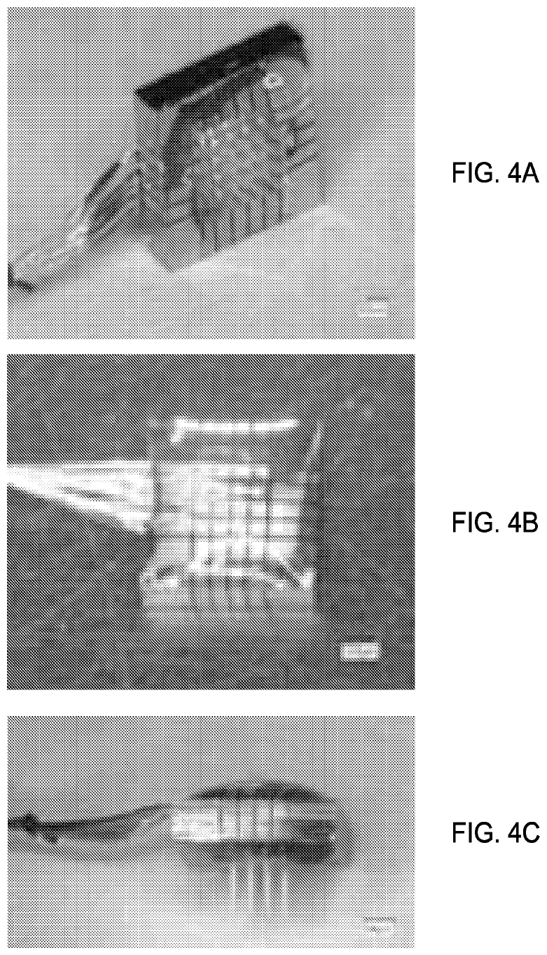

A 10×10 microprobe guide array can be designed and printed to fit over the μWell tMEA die to provide a well-defined growing path to the neurons stored within the μWell. In this example, a probe structure is an array of polymer needle shanks that can sustain and direct the neurons' axon growth (FIG. 4A-4C). As shown in FIG. 4A, an integrated and bonded tMEA device is composed of a GT2 3D printed 4×4 probe array and a backplane of silicon circuits. Each probe is 40 um in diameter on the top and 150 μm in diameter at the bottom, and the length of the probe is 1.5 mm. The bundle of insulated wire is bonded to pads on the periphery of the device as can be seen in FIG. 4B, and the entire backside is encapsulated with silicone as shown in FIG. 4C. This microprobe guide array can then be attached to the μwell die, or directly formed on the μwell die.

In this example, the polymer probe array of the tMEA device can be printed using a nano-resolution 3D printer (Photonic Professional GT2, Nanoscribe). This equipment employs a two-photon polymerization method to selectively cure a polymer resin on a substrate to create the designed 3D structure. The equipment accepts a wide range of polymer resins and can be used with the designs created with standard computer aided design (CAD) software. In this example, a 10×10 array of such probes and a frame with the precise dimensions of the μWell array dies for easy alignment. For the printing process a suitable resin can be used on a cleaned glass substrate. To save fabrication time, the structure can be printed as a hollow, resin filled shell. As one alternative, the probe guides can be printed, molded, or deposited directly onto the microwell array.

The polymer probe array can fit the μwell array and can be filled with cell culture medium. While the 3D polymerization fabrication process is well established, the hydrogel-filled probes can be fragile. The printed polymer probes can be strong enough so that they can survive pneumatic insertion into the brain tissue by withstanding mechanical failures. These polymer probes may be subject to undesired bending during the actual implantation as well. In such cases, the probe's Young's modulus or design (wall thickness, probe shape, etc.) can be adjusted to decrease fragility. The methods to stiffen the probe walls can also include freezing or coating these probes, for example with poly(ethylene glycol) and the like.

The immune response to implanted neurons can be avoided by implanting autologous neural stem cells or iPS cells. In addition, supporting biomaterials, such as extracellular matrix (ECM) hydrogel, can be loaded in tMEA's hollow probes. These supporting biomaterials provide three-dimensional (3D) structural support to neurons, and the projection of their axons. Chemicals or inhibitors for preventing inflammation and glial scarring can also be loaded along with the hydrogel.

A functional tMEA requires that at least a portion of the integrated neuronal cells can survive in the tMEA until implantation. Different neural stem cell types differ in their tolerance, characteristics and fates to the tMEA's physical environment. Various neural stem cell types (e.g., embryonic neural stem cells, cortical neural progenitor cells, or iPS) may be used.

As previously mentioned, the integrated neurons can develop by projecting its axon into the 3D printed polymer probes. In this case, standard resin can be used for 3D nanoscribe printing. As an example, the fabrication of the μWell chip can be followed by a pipette-loading of neuronal cells. The loaded neurons fall into the μWells. Since the dimensions of μWells can be designed and sized to hold only one single cell, each μWell can be filled by one single neuron after several cell loading-washing cycles. Extra cells will be washed away. This can be followed by printing the polymer probes to get the complete tMEA chip. The chip can then be connected to outside recording systems.

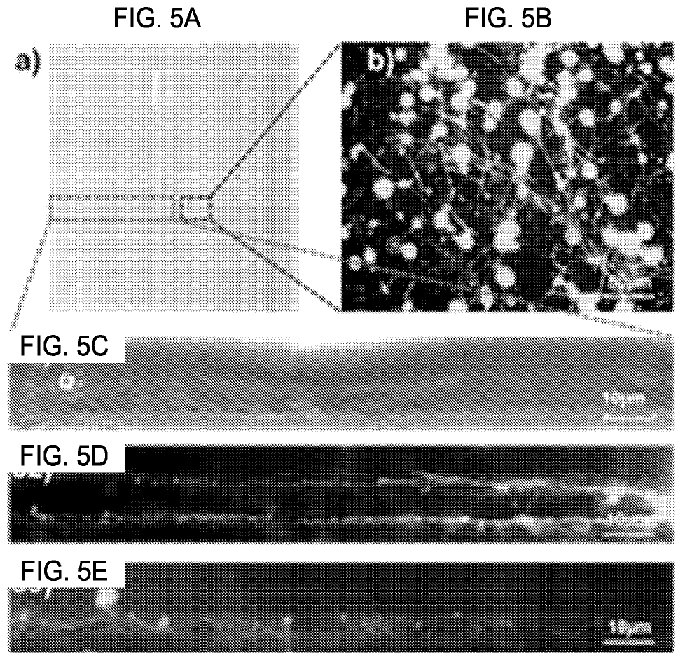

FIG. 5A-5E depicts the neuron's neurite initiation, axonal growth and projection process after being cultured in gelatin methacryloyl hydrogel. The initiation of neurite growth in hydrogel can occur in one day, and then the axon projected along the hydrogel probes during the subsequent days of culturing (FIG. 5C). Calcein-AM staining revealed that these axons' projection extended through the entire hydrogel probes (FIG. 5D-E). Moreover, to further increase the viability of the integrated neuronal cells, chemicals and/or growth factors can be additionally loaded along with the hydrogel to promote the projections.

To estimate development among integrated neurons within tMEA, a spike analysis can be applied to analyze the collected neural activities including extracellular action potentials (“spikes”) and groups of action potentials (“bursts”). In addition, except for recording the simultaneous neural activity, the tMEA is also capable of actively introducing electronic stimuli to trigger evoked responses (“evoke spikes/bursts”) from the integrated neurons in tMEA device. Briefly, the electrode, which contacts the neurons in the culture chamber of tMEA, was selected, a stimulus of +800 mV/200 μs per phase was applied to that electrode and was repeated every 10-15 s for 60 stimuli in each phase.

There can be artifacts from dendrites and/or synaptic contacts of the integrated neuronal cells in tMEA, but bidirectional (implanted-neuron-to-target-neuron synapses and especially target-neuron-to-implanted-neuron synapse) and multiple neural connections (axondendritic, axosomatic, and axoaxonic synapses) can occur. The formation of full-type synapses helps the functional integration of the implanted neurons and enables an accurate recording. Moreover, the dimension of each μwell (20 μm³) is designed and fabricated close to the size of a single neuronal cell (15˜20 μm in diameter). Therefore, a tight contact of neuron and μwell thus provide good neural signal recording from the neuronal cell to the recording spots in the μwell. Further, a gold-coated μwell with the Poly(1-lysine) (PLL) based solution can improve signal transfer. The PLL molecules preferentially form a self-assembled-monolayer on the Au electrode, thereby facilitating the attachment of the neuron cell on the functionalized electrode surface of the μwell.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A transition microelectrode, comprising:
a) a microwell array including a plurality of microwells having microwell openings;
b) a plurality of neuronal soma oriented within the plurality of microwells;
c) a bioerodible probe guide oriented over at least a portion of the microwell openings of the microwell array; and
d) an electrode electrically connected with the plurality of microwells.

2. The microelectrode of claim 1, wherein the plurality of microwells are oriented in a 2D array having the microwell openings oriented toward the bioerodible probe guide.

3. The microelectrode of claim 2, wherein the 2D array is a regular array having 36 to 1600 microwells.

4. The microelectrode of claim 1, wherein a single neuronal soma is oriented within each of the plurality of microwells.

5. The microelectrode of claim 4, wherein the neuronal soma is a neuronal stem cell.

6. The microelectrode of claim 5, wherein at least one of:
the neuronal soma arises from a genome of a subject in which the microelectrode will be implanted;
the neuronal soma all have a common genetic profile; and
the neuronal soma are of heterogeneous genetic profiles.

7. The microelectrode of claim 1, wherein the plurality of microwells are sized to accommodate a single neuronal soma.

8. The microelectrode of claim 1, wherein the plurality of microwells each have a width from 3 μm to 100 μm.

9. The microelectrode of claim 1, wherein the plurality of microwells include a conductive metal coating on an inner surface.

10. The microelectrode of claim 1, wherein the plurality of microwells further include extracellular matrix hydrogel within the microwells.

11. The microelectrode of claim 1, wherein the bioerodible probe guide has a conical shape extending from the microwell array.

12. The microelectrode of claim 1, wherein the bioerodible probe guide is formed of at least one of a biodegradable polymer and a meltable material.

13. The microelectrode of claim 1, wherein the bioerodible probe guide further comprises a rigidity enhancing coating, wherein the rigidity enhancing coating is optionally ice.

14. The microelectrode of claim 1, wherein the bioerodible probe guide has a tapered profile with a probe tip.

15. The microelectrode of claim 14, wherein the probe tip has an opening, permitting axons from the soma to extend outside of the opening.

16. The microelectrode of claim 1, wherein at least one of the plurality of microwells and the bioerodible probe guide further comprises inhibitors, anti-inflammatory agents, and growth factors.

17. The microelectrode of claim 1, wherein the microwells are coated with a biofilm layer that provides at least one of: nourishment for the plurality of soma, binding proteins to facilitate attachment of the soma to the microwell, proteins to prevent oxidative stress from stimulation, and proteins to aid in cell differentiation.

18. The microelectrode of claim 1, wherein the plurality of microwells have amorphous indentations to facilitate attachment of the soma to the plurality of microwells.

19. The microelectrode of claim 1, further comprising a signal processor unit operatively connected to receive electrical signals from the electrode, wherein the signal processor unit is capable of receiving information including measurements and an identifiable signature identifying the electrode as source of the information.

20. The microelectrode of claim 1, wherein the electrode is a composite electrode comprising multiple electrodes, each corresponding to a subset of soma.

21. The microelectrode of claim 20, wherein each soma within the subset contributes equally to its corresponding electrode's reading.

22. The microelectrode of claim 20, wherein each soma contributes differentially to its corresponding electrode's reading.

23. The microelectrode of claim 20, wherein the electrodes are each individually addressable.

24. A transition microelectrode array comprising an electrode array having a plurality of the transition microelectrodes of claim 1.

25. The array of claim 24, wherein the electrode array is a 2D array.

26. The array of claim 24, wherein the plurality of transition microelectrodes are separated by an electrically insulating material.

27. The array of claim 26, wherein the electrodes of each transition microelectrode are individually addressable.

28. A method of interfacing with neural tissue of a subject, comprising:
a) inserting a transition microelectrode array of claim 24 into a region of neural tissue;
b) allowing axonal projections from the plurality of soma to interface with native cells of the neural tissue outside the probe guide;
c) electrically communicating with native cells via the axonal projections to stimulate and/or record from the native cells; and
d) recording signals from the plurality of axons.

29. The method of claim 28, wherein each soma projects exactly one axon.

30. The method of claim 28, wherein the axonal projections travel through a cavity defined by the probe guide.

31. The method of claim 28, wherein the axonal projections are aided by growth factors.

32. The method of claim 28, wherein the axonal projections are capable of selectively and/or promiscuously binding to the native cells.

33. The method of claim 32, wherein the selective and/or promiscuous binding is determined by inclusion of specific guidance molecules and/or by a manipulated genome of the soma.

34. The method of claim 28, wherein the axonal projections interface with native neurons in a form of various synapses, including electrical, chemical, mossy, filopodial, and en passant.

35. The method of claim 28, wherein the stimulating includes release of neurotransmitters for inhibition and/or excitation.

36. The method of claim 28, further comprising stimulating the plurality of axons using a variable input to activate a variable number of soma.

37. The method of claim 36, wherein the variable input causes spike patterns including one or more of standard tonic, bursting, adaptive, and delayed.

38. The method of claim 28, wherein the recording results from retrograde signaling from the axon to the soma.

39. The method of claim 28, wherein each electrode receives a plurality of recordings.

40. The method of claim 28, wherein the stimulations from the native cells are measured as voltage signals that are variable and continuous rather than binary.
